(19) United States
(12) Patent Application Publication
(10) Pub. No.: US 2016/0223546 A1
(43) Pub. Date: Aug. 4, 2016

(54) SYSTEMS AND METHODS FOR THE MOLECULAR QUANTIFICATION OF SKIN DAMAGE IN SKIN

(71) Applicant: Ronald MOY, Beverly Hill, CA (US)

(72) Inventor: Ronald L. Moy, Beverly Hills, CA (US)

(21) Appl. No.: 15/021,260

(22) PCT Filed: Sep. 11, 2014
(86) PCT No.: PCT/US14/55230

§ 371 Date: Mar. 10, 2016

Related U.S. Application Data
(60) Provisional application No. 61/876,554, filed on Sep. 11, 2013.

(51) Int. Cl. G01N 33/574

(52) U.S. Cl. CPC G01N 33/5743; G01N 2800/52; G01N 2800/50

(57) ABSTRACT

Systems and methods for quantifying skin damage, such as oxidative skin damage from sun exposure, are disclosed herein. The skin damage can be quantified using by using a proximity-based assay that measures a skin marker, such as peptide 3R. The quantitative data can be advantageously used to serve, for example, as markers of overall dermatologic health, risk stratification factors, markers for the presence of dermatologic conditions, including clinical disease, predictors of subclinical and/or minimal residual disease presence, determinants of treatment response and disease progression, and prognosticators of patient outcome.

### PRIORITY CLAIM

This application claims the benefit under 35 U.S.C. §119(e) as a nonprovisional application of U.S. Prov. App. No. 61/876,554 filed on Sep. 11, 2013, which is hereby incorporated by reference in its entirety.

### BACKGROUND

Immunohistochemistry (IHC) and fluorescence in situ hybridization (FISH) are the currently available methods of detecting the presence of proteins or genes in tissue such as the skin, but neither provides an actual quantity of the target protein, limiting their usefulness. What is needed are systems and methods for quantitatively measuring a skin marker, which could have a wide variety of diagnostic as well as therapeutic applications.

### SUMMARY

Disclosed herein are systems and methods of quantitatively measuring a skin marker, for a wide variety of diagnostic as well as therapeutic applications, including but not limited to assessing skin damage and initiating and/or changing prophylactic and/or therapeutic measures for a particular individual (e.g., initiating or changing sun-protection measures); determining an individual's risk of developing a particular dermatologic condition; diagnosing a particular dermatologic condition; or assessing therapeutic effect of a particular dermatologic condition in some embodiments.

In some embodiments, a method of determining a patient's risk of developing a dermatologic condition is disclosed. The method can include, in some embodiments, any number of the following steps: receiving a skin biopsy sample of the patient, the skin biopsy sample comprising a skin marker (e.g., peptide 3R); providing a first binding partner conjugated to biotin, wherein the first binding partner is a first antibody; providing a second binding partner conjugated to a fluorescent reporter tag, wherein the second binding partner is a second antibody; exposing the first binding partner to the skin biopsy sample, such that the first binding partner binds to a first binding site of the skin marker (e.g., peptide 3R); exposing the second binding partner to the skin biopsy sample, such that the second binding partner binds to a second binding site of the skin marker (e.g., peptide 3R); exposing the skin biopsy sample to a molecular cleavage element such that the molecular cleavage element binds to the first binding partner, the molecular cleavage element comprising a photosynthesizer; illuminating the tissue sample with light such that the molecular cleavage element causes the fluorescent reporter tag to dissociate from the second binding partner; and quantitatively determining the amount of the fluorescent reporter tag released.

Also disclosed herein is a method of determining the effect of therapy of a dermatological condition. The method can include, in some embodiments, any number of the following steps: receiving a first skin biopsy sample of the patient, the first skin biopsy sample comprising a skin marker, wherein the skin marker comprises peptide 3R; providing a first binding partner conjugated to biotin, wherein the first binding partner is a first antibody; providing a second binding partner conjugated to a fluorescent reporter tag, wherein the second binding partner is a second antibody; exposing the first binding partner to the first skin biopsy sample, such that the first binding partner binds to a first binding site of peptide 3R; exposing the second binding partner to the first skin biopsy sample, such that the second binding partner binds to a second binding site of peptide 3R; exposing the skin biopsy sample to a molecular cleavage element such that the molecular cleavage element binds to the first binding partner, the molecular cleavage element comprising a photosynthesizer; illuminating the tissue sample with light such that the molecular cleavage element causes the fluorescent reporter tag to dissociate from the second binding partner; and quantitatively determining the amount of the fluorescent reporter tag released to determine the amount of peptide 3R present in the first skin biopsy sample. The method can also include, in some embodiments, any number of the following steps: receiving a second skin biopsy sample of the patient, the second skin biopsy sample comprising a skin marker, wherein the skin marker comprises peptide 3R; determining the amount of peptide 3R present in the second skin biopsy sample; and comparing the amount of the peptide 3R present in the second skin biopsy sample with the amount of the peptide 3R present in the first skin biopsy sample, wherein the second skin biopsy sample was taken from the patient for a period of time, such as, for example, at least about a month, 3 months, 6 months, a year, or more following the first skin biopsy sample.

In some embodiments, disclosed herein is a method that can include any number of the following steps: performing a skin biopsy on a patient, wherein performing a skin biopsy comprises taking a skin biopsy sample comprising a skin marker, wherein the skin marker comprises peptide 3R; ordering a diagnostic test on the skin biopsy sample, wherein the diagnostic test comprises the steps of: receiving the skin biopsy sample of the patient; providing a first binding partner conjugated to biotin, wherein the first binding partner is a first antibody; providing a second binding partner conjugated to a fluorescent reporter tag, wherein the second binding partner is a second antibody; exposing the first binding partner to the skin biopsy sample, such that the first binding partner binds to a first binding site of peptide 3R; exposing the second binding partner to the skin biopsy sample, such that the second binding partner binds to a second binding site of peptide 3R; exposing the skin biopsy sample to a molecular cleavage element such that the molecular cleavage element binds to the first binding partner, the molecular cleavage element comprising a photosynthesizer; illuminating the tissue sample with light such that the molecular cleavage element causes the fluorescent reporter tag to dissociate from the second binding partner; and quantitatively determining the amount of the fluorescent reporter tag released to determine the amount of peptide 3R present in the skin biopsy sample; and analyzing the results of the amount of peptide 3R present in the skin biopsy sample. The method can also include the step of determining the risk of the patient developing a dermatologic condition, such as a skin cancer (e.g., melanoma, basal cell carcinoma, or squamous cell carcinoma in some embodiments). The method can also include the step of diagnosing a dermatologic condition, which can be, in some embodiments, selected from the group including melanoma, basal cell carcinoma, squamous cell carcinoma, actinic keratosis, xeroderma pigmentosum, albinism, xerosis, ichtyosis, keratoderma, dermatofibroma, dermatitis, acne, neurodermatitis, dermatitis herpetiformis, vitiligo, vasculitis, pemphigus, bullous pemphigoid, hyperkeratosis, eczema, psoriasis, rosacea, pityriasis rosea, warts, bacterial skin infection, viral skin infection, and a fungal skin infection. The method can also include the step of treating a dermatologic condition based upon the amount of peptide 3R present in the skin biopsy sample. The amount of peptide 3R present in the skin biopsy sample, in some embodiments, can be a first amount of peptide 3R present in a first skin biopsy sample, and also include the step of comparing the first amount of peptide 3R with a second amount of peptide 3R present in a second skin biopsy sample of the patient. The second skin biopsy sample can be separated by a period of time, such as at least about a week, a month, or more compared with the first skin biopsy sample. The method can also include the step of initiating therapy, modifying therapy, or discontinuing therapy for a dermatologic condition after the comparing step.

### BRIEF DESCRIPTION OF THE DRAWINGS

FIG. 1 illustrates steps in a quantitative proximity-based assay for determining the amount present of a skin marker in a patient's skin sample, according to some embodiments of the invention.

FIG. 2A is a flow chart illustrating possible steps in a quantitative proximity-based assay for determining the amount present of a skin marker in a patient's skin sample, according to some embodiments of the invention.

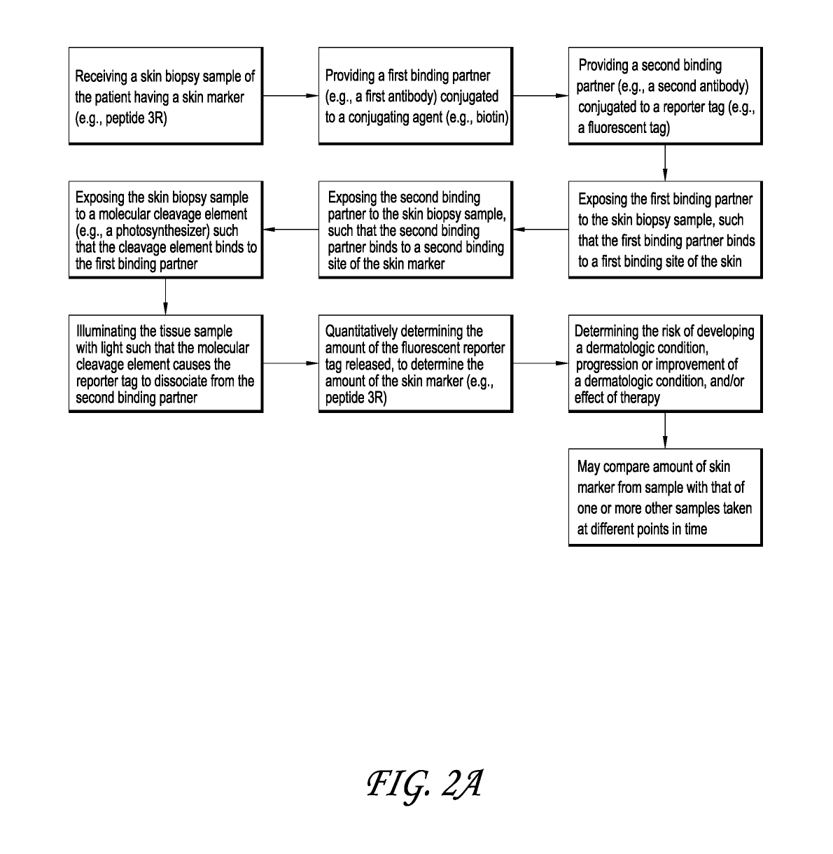

FIG. 2B is a flow chart illustrating possible diagnostic or treatment algorithms for a health care provider, based upon taking a skin biopsy and ordering a quantitative proximity-based assay for determining the amount present of a skin marker in a patient's skin sample, according to some embodiments of the invention.

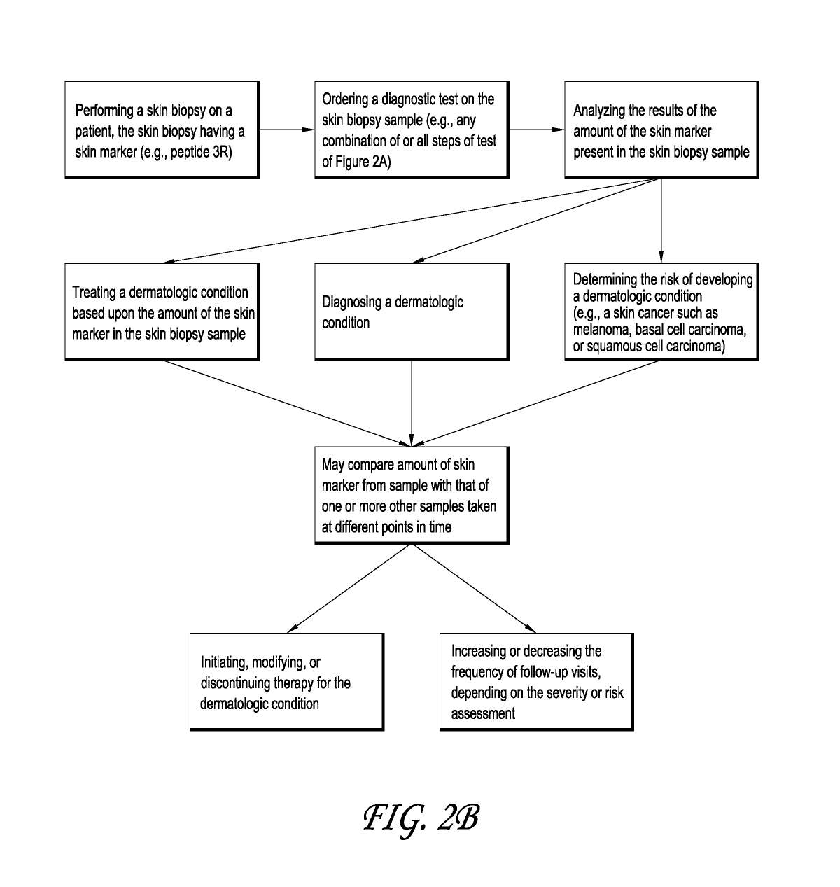

### DETAILED DESCRIPTION

In one embodiment of the invention, systems and methods to quantify skin damage, such as cumulative oxidative sun damage in a clinically meaningful way in human skin are disclosed. A peptide sequence, peptide 3R (described in detail below), has recently been discovered which could result from long-term sun exposure and aging of the skin. IHC histoscores do not provide detailed information about the amount of the target protein, which is not noted by IHC until sometimes the late 50s in age in humans However, as sun damage is a continuous process beginning at birth, these markers could be progressively accumulating which require a more sensitive and/or specific test to measure and quantify. For example, a test for a breast cancer protein was recently developed which has shown increased sensitivity and specificity for the target protein of a human monoclonal antibody therapy. This test yields an actual number of receptors on breast cancer cell surfaces rather than merely a more vague qualitative description. Directing, for example, this new proximity-based immunoassay towards a skin marker, e.g., a skin marker of sun damage can measure oxidative damage in skin. Such a test could quantify the marker of sun damage to a clinically distinguishable degree.

There are currently no known quantitative assays that measure oxidative damage in the skin, but an ever-expanding arsenal of lasers, chemical peels, vitamin A derivatives, DNA repair creams, and even recombinant growth factors are now being sold with promises of reversing years of sun damage and preventing skin cancer. Producing the appearance of youthful skin, however, may not mean that years of ultraviolet damage have been reversed. With the use of quantitative measurements as disclosed herein, an actual score in terms of improvement could be provided to the patient and the treating physician. Furthermore, a response to therapy could be evaluated; for example, a first patient could respond well to a particular therapy, such as DNA-repair creams for example, while a second patient might not respond well to the same therapy. Rather than spending time and financial resources on a treatment that they might not respond to, the second non-responder patient could try an alternative. Skin cancer risk can be more accurately quantified and current and future therapies could be tested, and risk reduction of that particular therapy could be quantified as well. Cumulative oxidative damage and cancer risk could be correlated to skin type. Surgeons could decide to be more aggressive in skin with more damage as determined by sampling from a biopsy. Studies could determine how often skin exams are needed by correlating risk with molecular cumulative damage measures, or acceleration in those values. The actual “sun years” prevented by sunscreen use could be calculated, the type of sun protection can be prescribed or modified, and permanent damage from a single sunburn determined For example, a 40 year old patient could be told they have the cancer risk of the average 60 year old if an accurate molecular measure of cumulative sun damage predicted it, and change habits accordingly. Using diagnostic systems and methods as disclosed herein, our skin could be like a UV dosimeter, tallying the exposure throughout our lifetime, and determining whether therapy reverses or at least slows down the damage. These are all nonlimiting potential uses for a reliable, dynamic molecular assay of sun damage in skin.

Aging in human skin has two components: (1) chronological aging, which is genetically determined, and (2) extrinsic aging, which is due mainly to UV irradiation and the resulting reactive oxygen species (ROS). ROS in the dermis cause a loss of collagen and accumulation of disorganized elastic fibers, as well as DNA damage. Therefore, a measure of cumulative oxidative damage in skin would be meaningful. However, studies comparing young and aged human skin have indicated that the levels of very few proteins are changed between the two groups. At the transcriptional level, there is very little change in gene expression.

Much work has been done identifying markers of UV-induced and oxidative stress-induced damage in skin, but these markers change too dramatically in response to UV exposure to provide meaningful long-term significance. Several markers have been used previously to quantify this cumulative damage in clinical research studies. However, many of these markers, including gamma-phosphorylated histone H2AX (which repairs double-stranded DNA breaks), and oxidized deoxyguanosine (a marker of oxidative stress), also increase acutely following UV irradiation, and through a variety of mechanisms quickly return to baseline levels. While these markers, for example, could be used in conjunction with certain embodiments of the invention and do accumulate throughout life in response to environmental insults, the day-to-day variability could potentially make them difficult to use reliably. For example, if a patient has been at the beach prior to coming into clinic, they could potentially have drastically increased levels of the gamma-H2AX and oxidized deoxyguanosine compared to the same patient only hours earlier. Advantageous protein markers for sun or age damage could show accumulation in a more stable manner D-aspartate (Asp) is a biologically rare amino acid residue and can be formed by racemization of the Asp amino acid during chronological aging and accelerates with UV exposure, making it a good marker for cumulative oxidative damage. Because only the native L-form of amino acids can be used during protein synthesis, the D-form is not usually present in metabolically active tissue. D-Asp can be found in teeth, the lens of the eye, aorta, and the brain in elderly humans The D-beta-Asp residue was recently found in skin as well. In some embodiments of the invention, a polyclonal antibody has been synthesized to identify three D-beta-Asp repeats in positions 149-153 of the human protein alpha-crystallin (found in the lens) that can be quantitatively measured. The antibody was directed against the peptide Gly-Leu-D-beta-Asp-Ala-Thr-Gly-Leu-D-beta-Asp-Ala-Thr-Gly-Leu-D-beta-Asp-Ala-Thr (referred to herein as “peptide 3R”). The aforementioned antibody could distinguish the configuration of Asp-residue, reacting strongly only with the D-beta-Asp containing peptides, and not with L-alpha-Asp, L-beta-Asp, nor D-alpha-Asp. In the lens, the D-beta-Asp residue was only observed with immunohistochemical staining in aged eyes, and not in young eyes. D- and beta-isomers in protein correlate with both aging and sun exposure. Besides the eye, skin is another organ which experiences significant UV damage. Sun-damaged skin in elderly humans shows accumulation of fragmented dermal elastic fibers, called actinic or solar elastosis. The antibodies to peptide 3R were detected only in sun-exposed skin of older study participants, and not in sun-exposed skin of children. D-beta-Asp peptide-containing protein is accelerated by sunlight exposure and accumulates in aged skin due to sun exposure. It was determined that the peptide 3R was part of elastin. Further details regarding Peptide 3R can be found, for example, in Fujii et al., Biochem. Biophys. Res. Comm , 294 (2002) 1047-1051, which is hereby incorporated by reference in its entirety.

Peptide 3R, for example, is a good marker of cumulative sun-induced oxidative damage because it is detectable prior to the appearance of elastotic tissue by histopathological examination. The immunoreactive elastic fibers appeared in the mid-dermis prior to the visualization of the distinctive elastotic fibers of actinic elastosis, implying that intact elastic fibers undergo abnormal racemization before resulting in the abnormal actinic elastotic materials. Additionally, immunoreactive fibers were seen in sun-protected areas from elderly skin (over 65 years) in the middle and lower dermis, suggesting an element of chronological aging as well. The mechanism of racemization of L-alpha to D-beta-Asp is thought to come from a spontaneous nonenzymatic chemical reaction where long-term UV irradiation or constant oxidative stress is present. Since elastin has a very slow turnover, it is more susceptible to racemic modification.

The same antibody against peptide 3R is also detectable in UV-damaged epidermis. D-beta-Asp containing proteins in the epidermis of UVB-irradiated mice, identified as members of the keratin family of proteins, are further support for its use as a marker of sun-damaged skin. Additional studies by others have showed that in isolated elastin-mimic peptides, ultraviolet exposure alone did not increase L- to D-Asp conversion. When instead a free radical was added, racemization did increase significantly. It has been concluded that the generation of reactive oxygen species accelerates the conversion of L- to D-Asp in skin elastin. It is important to point out that this experiment was performed on simple peptide chains representing part of elastin, not actual skin. In normal skin, UV irradiation produces free radicals.

In one embodiment of the invention, an assay to quantitatively measure a skin marker, such as peptide 3R, is used to quantify the presence of the peptide in skin tissue. For example, a VeraTag™ quantitative proximity-based assay (Monogram Biosciences, South San Francisco, Calif.) can be used to quantify the D-aspartyl-containing peptide 3R presence in skin tissue. VeraTag™ was recently developed to quantify HER2 protein expression. The HER2/neu/erbB2 family of receptor tyrosine kinases are components of signal transduction pathways that are amplified or overexpressed in approximately 30% of breast cancers, and although the HER2 antibody Herceptin (traztuzumab) has had encouraging results, only about 50% of HER2 positive metastatic breast cancer patients respond in a clinically significant way.

The two commercially available methods for testing HER2 status in breast cancer are immunohistochemistry (IHC), which detects HER2 protein expression levels, and fluorescence in situ hybridization (FISH), which detects amplified HER2 genes. IHC gives a semi quantitative measure of protein levels, and FISH, while giving a quantitative measure of gene amplification, does not always correlate with protein expression. Neither method gives quantitative data which would completely predict a response to therapy.

However, the VeraTag™ assay is able to quantify HER2 proteins using HER2 antibodies conjugated to fluorescent tags that are released, collected, and counted by a mechanism described in Example 1 below. In one embodiment, antibodies to the target skin marker (e.g., Peptide 3R) will be attached to two different quantitative proximity-based assay molecules. One comprises a fluorescent tag; a second contains a treated photo-labile biotin. When the two different antibody-VeraTagTm complexes bind adjacent HER2 proteins, subsequent exposure to a light source (e.g., at or about 670 nm) creates an unstable reactive oxygen molecule from the biotin, which cleaves a thioether bond between the fluorescent tag and anti-HER2 antibody nearby. The fluorescent tag is collected and quantified, indicating total HER2 protein level. The HER2 protein expression levels from samples of 170 breast tumors correlated with IHC test categories (P<0.001) at both high and low levels, but additionally were continuous and overlapped the IHC categories, and gave a dynamic range of up to 10 times the IHC histoscores at higher HER2 levels, prompting the study of the VeraTag™ assay as a predictive marker for traztuzumab therapy.

Since not all patients selected for traztuzumab therapy respond, the limitations of IHC and FISH become apparent, and highlight the advantage of a VeraTag™ assay. A recent study looked at 102 patients previously assessed at IHC 3 positive, or IHC 2 positive and FISH positive previously treated with traztuzumab. Fourteen percent of FISH-negative patients had high levels of HER2 protein by VeraTag™ assay (discordant HER2 total expression high), and 13% of FISH-positive patient had low levels of HER2 protein (discordant HER2 expression low). Time to progression was increased in the discordant HER2 positive group, and decreased in the discordant HER2 negative group, indicating that subsets of patients can test positive by both IHC and FISH but have low levels of HER2 and thus have poor responses to traztuzumab, while another subset can test negative for IHC and FISH (suggesting poor treatment response), but actually have high levels of HER2 and respond well. Treatment response was most related to total HER2 protein levels (as quantified by VeraTag™ assay), rather than IHC and FISH.

D-aspartyl residues have been noted to accumulate in human skin with aging, oxidative stress, and UV exposure. Since oxidative damage affects DNA as well as protein, a stable marker of oxidative damage could be very useful to the field of dermatology. While the VeraTag™ assay is used commercially in the aforementioned vastly different field of measuring the specific HER2 breast cancer marker, in some embodiments of the invention, it has been surprisingly discovered that using a quantitative proximity-based assay such as, for example, the VeraTag™ assay could better quantify one, two, or more markers of a skin condition, e.g., sun damage compared to standard IHC.

Therefore, in some embodiments of the invention, disclosed are systems and methods of quantitatively determining the health of a patient's skin, determining the amount of damage, such as sun damage, of a patient's skin, determining a patient's risk of developing a particular dermatologic condition, determining whether a patient has a particular dermatologic condition, determining whether prophylactic or a therapeutic regimen for a particular dermatologic condition should be initiated, and/or assessing the efficacy of a particular therapy to a particular dermatologic condition, and thus adjusting, switching, or discontinuing the therapy, or adding additional therapies based on a single or multiple serial quantitative determinations. The dermatologic condition could be, for example, a skin cancer such as melanoma, basal cell carcinoma, or squamous cell carcinoma; a condition associated with an increased risk of developing skin cancer, or a non-cancerous condition. The therapy could be, for example, a systemic or local therapeutic agent, e.g., one, two, or more drugs or biologic agents, including sunscreen preparations; a physical or mechanical skin barrier, such as sunglasses or sun-shielding clothing, headwear, or footwear for example; an energy-based therapy such as infrared, visible, or ultraviolet laser or light; ultrasound, RF, microwave, cryotherapy, radiation therapy, or surgery, or any combination of the foregoing. In some embodiments, the therapy adjustment could involve adding sunscreen or changing the type of sunscreen or increasing the sun protection factor (SPF) of sunscreen recommended for the particular patient based upon one or serial quantitative assessment(s) of the patient's skin health, e.g., the amount of sun damage, in order to prevent or reduce future DNA damage in the skin.

In one embodiment, the method includes providing a skin biopsy sample of the patient. The skin biopsy sample comprises a skin marker, for example, peptide 3R. A first binding partner, such as an antibody, is provided that is conjugated to a conjugating agent, such as biotin. A second binding partner, such as an antibody, is then provided that is conjugated to a reporter tag, such as a fluorescent reporter tag. The first binding partner and the second binding partner are then exposed to the skin biopsy sample. The first binding partner and second binding partner bind (e.g., selectively) to binding sites on peptide 3R in some embodiments. One or more of the binding sites on peptide 3R could be, for example, one, two, or more D-beta-Asp residues. The skin biopsy sample is then incubated with a molecular cleavage element (e.g., streptavidin or avidin bound to a photosensitizer, such as methylene blue). The streptavidin or avidin will then bind to the biotin of the first binding partner, in turn binding the molecular cleavage element. The tissue sample is then exposed to an activator of the molecular cleavage element, such as light, such that the molecular cleavage element causes the fluorescent reporter tag to dissociate from the second binding partner. The fluorescent reporter tag can then be quantitatively measured after being separated, such as via electrophoresis (e.g., capillary electrophoresis, chromatographic separation, or mass spectrometry.

In one embodiment, a system or kit for quantitatively determining the health of a patient's skin, determining the amount of damage, such as sun damage, of a patient's skin, determining a patient's risk of developing a particular dermatologic condition, determining whether a patient has a particular dermatologic condition, or assessing the efficacy of a particular therapy to a particular dermatologic condition, and adjusting, changing, or discontinuing the therapy based on a single or multiple serial quantitative determinations includes a first binding partner that includes a reporter tag, the first binding partner configured to bind to a binding site of a skin marker, such as peptide 3R, and a second binding partner that includes a molecular cleavage element configured to cleave the reporter tag from the first binding partner, the second binding partner configured to bind to a binding site of a skin marker, such as peptide 3R. The system could also include an activator of the molecular cleavage element, and one, two, or more skin samples from the patient.

### Skin Markers

A variety of skin markers can be quantitatively measured with the systems and methods disclosed herein. In some embodiments, the markers may be, but are not limited to cells, peptides, (including polypeptides), proteins, carbohydrate markers, or nucleic acids. In some embodiments, the skin marker comprises alpha-crystallin, or a portion thereof, such as a peptide sequence thereof; peptide 3R or a portion thereof; an isolated D-beta Asp sequence; a plurality, e.g., two, three, or more consecutive D-beta-Asp repeats or repeats within about 20, 15, 10, 8, 6, or less amino acids between each other. In some embodiments, the skin markers include repeats of Gly-Leu-D-beat-Aso-Ala-Thr in positions 149-153 of human alpha-crystallin.

### Skin Marker Binding Partners

In some embodiments, nucleotides (e.g., oligos, LNAs (locked nucleic acids), PNAs (peptide nucleic acids), cDNA, nucleic acid probes, chromatographic affinity probes or fragments thereof or their derivatives, complementary fragments or larger) antibodies (e.g., monoclonal, polyclonal, FAb fragments, etc.), proteins, or any biological or synthetic material (e.g., biotin-avidin) that is complementary or substantially complementary to one, two or more binding sites on the skin marker in question and can be assessed in vivo or ex vivo can be skin marker binding partners. The desired binding partner(s) are capable of binding a target binding site of the corresponding target skin marker of interest in a sufficient concentration and manner that permits retrieval for qualitative or quantitative analysis of the marker.

In some embodiments, binding partners are antibodies that are configured to bind to a target binding site on alpha-crystallin, peptide 3R, or a portion thereof. The target binding site could be, for example, a portion of alpha crystallin, such as a peptide sequence thereof; peptide 3R or a portion thereof; or one, two, three, or more D-beta-Asp repeats in close proximity to each other, e.g., three D-beta-Asp repeats (e.g., repeats of Gly-Leu-D-beat-Aso-Ala-Thr) in positions 149-153 of human alpha-crystallin. Preparation of one such antibody is described, for example, in Fujii et al., Mol. Vision 2000; 6:1-5 which is hereby incorporated by reference in its entirety.

In some embodiments, a first antibody is conjugated to a conjugating agent, such as biotin. The biotin binds a molecular cleavage agent having a component having a high affinity to the conjugating agent. For example, streptavidin and avidin have a high affinity for biotin. The molecular cleavage agent could be, for example, a photosensitizer. Sensitizer molecules can be conjugated to an antibody by various methods and configurations. For example, an activated sensitizer, such as e.g., methylene blue or phthalocyanine, activated with e.g., NHS ester, aldehyde, or sulfonyl chloride, can be reacted with the amino groups in antibodies. These conjugates can then be used directly in various assays. Also, multiple activated sensitizer molecules can be coupled with antibody, e.g. by using an aminodextran-sensitizer conjugate containing 20-200 sensitizers and 200-500 amino groups, coupled to periodate-oxidized antibody molecules, generating an antibody-dextran-sensitizer conjugate. In some embodiments, the sensitizer conjugates will be generated using purified mouse monoclonal antibody 12CA5, which recognizes the amino acid sequence YPYDVPDYA, specific for hemagglutinin, from Roche Diagnostics, Indianapolis, Ind. coupled to methylene blue activated with NHS ester, to generate methylene blue-conjugated mouse anti-HA.

A second antibody is conjugated to a reporter tag, which can be a fluorescent reporter tag in some embodiments. Various reporter tags that can be used with embodiments of the invention are described in U.S. Pat. No. 7,105,308 to Chan-Hui et al.

### Diseases

In some embodiments, the skin markers quantified could serve, for example, as markers of overall dermatologic health, risk stratification factors, markers for the presence of dermatologic conditions, including clinical disease, predictors of subclinical and/or minimal residual disease presence, determinants of treatment response and disease progression, and prognosticators of patient outcome. When determining treatment response and disease progression, skin samples containing skin markers can be obtained in some embodiments, at least one day, one week, two weeks, one month, two months, three months, six months, a year, two years, or more apart in time from each other. After quantitatively determining the level of skin marker present, a report could be generated with normal and abnormal range values. A physician could then determine whether or not to initiate, modify, discontinue treatment, or change the frequency of patient monitoring based on the quantitative level of skin marker, or based on a series of levels measured over time.

In some embodiments, the dermatologic condition could be a malignant condition such as a skin cancer, including melanoma, basal cell carcinoma, or squamous cell carcinoma. The dermatologic condition could predispose a patient to a greater risk of developing skin cancer, the condition includes one or more such as actinic keratosis, xeroderma pigmentosum, or albinism, for example. A wide variety of other dermatologic conditions, including but not limited to xerosis, ichtyosis, keratoderma, dermatofibroma, dermatitis, acne, neurodermatitis, dermatitis herpetiformis, vitiligo, vasculitis, pemphigus, bullous pemphigoid, hyperkeratosis, eczema, psoriasis, rosacea, pityriasis rosea, warts; bacterial, viral, fungal, or other infections can also be diagnosed, risk stratified, or therapy modified using the systems and methods as disclosed herein. In some embodiments, the dermatologic condition could be a manifestation of a systemic disease, such as an autoimmune disease such as systemic lupus erythematosus, scleroderma, or rheumatoid arthritis, for example.

### Skin Samples

In some embodiments, the skin sample could be obtained via one, two, three, four, five, or more shave biopsies, punch biopsies, or incisional or excisional biopsies, or a combination of the above. In some embodiments, the skin sample comprises, for example, elastin and/or collagen. The skin sample could be obtained from any desired anatomical location on the body, including one or more of the scalp, face, neck, chest, back, buttocks, upper or lower extremities, or genitalia for example. The skin sample could have a depth of no more than about 20 mm, 15 mm, 10 mm, 8 mm, 6 mm, 5 mm, 4 mm, or less in some embodiments. The skin sample can include one or more layers of the epidermis, dermis, and hypodermis.

### Separation of Reporter Tags

Molecular tags can be designed for separation by a separation technique that can distinguish molecular tags based on one or more physical, chemical, and/or optical characteristics (referred to herein as “separation characteristics”). Separation techniques that may be used with the various embodiments include normal phase or reverse phase HPLC, ion exchange HPLC, capillary electrochromatography, mass spectroscopy, gas phase chromatography, and the like. In some embodiments, the separation technique selected is capable of providing quantitative information as well as qualitative information about the presence or absence of molecular tags (and therefore, corresponding analytes). In one aspect, a liquid phase separation technique is employed so that a solution, e.g. buffer solution, reaction solvent, or the like, containing a mixture of molecular tags is processed to bring about separation of individual kinds of molecular tags. Usually, such separation is accompanied by the differential movement of molecular tags from such a starting mixture along a path until discernable peaks or bands form that correspond to regions of increased concentration of the respective molecular tags. Such a path may be defined by a fluid flow, electric field, magnetic field, or the like. The selection of a particular separation technique depends on several factors including the expense and convenience of using the technique, the resolving power of the technique given the chemical nature of the molecular tags, the number of molecular tags to be separated, the type of detection mode employed, and the like. In some embodiments, molecular tags are electrophoretically separated to form an electropherogram in which the separated molecular tags are represented by distinct peaks.

### EXAMPLE 1

One embodiment of a method of diagnosing a method of quantifying skin damage of one, two or more patients, or a selected patient population that could be used will now be described. After giving informed written consent, a patient undergoes one, two or more punch biopsies (e.g., about 6 mm); one or more from a sun exposed area and/or one or more from a sun-protected area. Punch biopsies can cause minor pain and have a small risk of bleeding, infection, and some scarring. The patient's age, gender, Fitzpatrick skin type, skin sample location, and tissue type (tumor, benign skin, etc.) are then recorded. Patients will be asked to describe any recent sun exposure within the last 48 hours. Additionally, patients will also be asked to describe their lifetime cumulative sun exposure. This generally provides an accurate estimate of lifetime sun exposure and is standardized for historical studies. In some embodiment, patients are selected in each in the following age divisions: 18-29, 30-39, 40-49, 50-59, 60-69, 70-79, and 80+. Thus, a baseline level of peptide 3R with age can be established. Because many patients are being seen for surgical procedures, one or more of the punch biopsies will be taken adjacent to the surgical procedure to minimize scarring whenever possible. Samples will be formalin-fixed and paraffin embedded (FFPE) into tissue blocks as known in the art. After histopathological examination, e.g., by a board certified dermatopathologist, the remainder of the FFPE blocks will be sent to Monogram Biosciences for VeraTag™ assay. If subjects are being seen for excision or biopsy of another lesion (benign or malignant), then tumor tissue, as well as unaffected skin from the surgical margin, and the punch biopsy specimens will all be subjected to the VeraTag™ assay to quantify peptide 3R content.

To detect peptide 3R, a pair of antibodies to the D-beta-Asp containing peptide present in photodamaged skin are attached to a fluorescent tag on one a first antibody, and to light-activated “molecular scissors” on a second antibody. When two anti-peptide 3R antibodies bind 3R peptides in close proximity, exposure to a customized LED (light emitting diode) “activates” the scissors attached to the second antibody, and the fluorescent tag on the first antibody is released, collected, and measured. Each fluorescent tag collected thus represents a pair of peptide 3R sequences.

The assay format is based on the proximity and light-dependent release of antibody-conjugated fluorescent reporter tags, with unique migration properties for capillary electrophoresis (CE). The specificity and sensitivity resulting from the required proximal binding of 2 antibodies, coupled with the analytical power of CE, provides the capability to detect molecular markers (peptide 3R) in formalin-fixed, paraffin-embedded (FFPE) skin samples. The proximity requirement for the assay signal release will enable the quantitative measure of D-beta-Aspartyl-containing peptides as surrogate markers of cumulative UV damage. Sections of 5 micrometers will be cut on a microtome, and section area (mm²) determined by scanning with a commercial flatbed scanner and estimated by ImageJ software. After the VeraTag™ assay, slides of tissue will be stained with hematoxylin and eosin (H & E), and the sample area calculated by a dermatopathologist.

Complete descriptions of the materials and methods that can be used, in some embodiments, of a skin marker quantitative assay are detailed in Shi et al., Diagn. Mol. Pathol. 2009; 18: 11-21, which is hereby incorporated by reference in its entirety. Other materials and methods that can be used in combination with systems and methods herein can be found, for example, in U.S. Pat. No. 7,105,308.

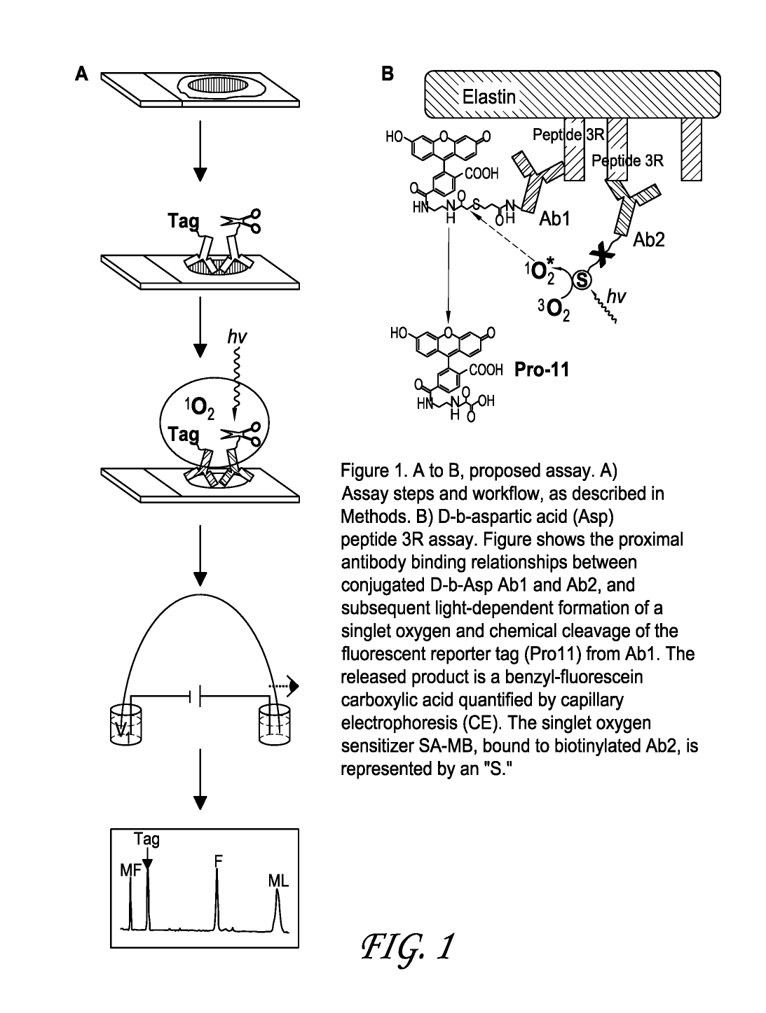

Polyclonal antibodies to D-beta-Asp in position 151 of alphaA-crystallin, designated as peptide 3R are attached to either fluorescent reporter group “tags” (e.g., Pro11) or biotin. Antibody-fluorescent tag and antibody-biotin conjugates will be made using sulfo-NHS-LC-LC-biotin (Pierce) according to manufacturer's protocol, and the conjugation products purified by high performance liquid chromatography (Agilent). The steps are as follows, represented in FIG. 1.

1) The fluorescent reporter tag-conjugated and the biotin-conjugated antibody bind to peptide 3R.

2) Incubation with streptavidin-conjugated photosensitizer methylene blue (SA-MB: “molecular scissors”) which is synthesized and purified according to the VeraTag™ protocol.

3) Illumination with light (e.g., at 670 nm), during which the photosensitizer bound to the biotin antibody converts dissolved oxygen to a reactive, singlet state oxygen (¹O₂) in buffer solution. The ¹O₂ molecules are short lived, so their limited diffusion (100 nm) results in proximity based cleavage of a thioether bond and release of the fluorescent tag from the tissue-bound antibody (FIG. 1B).

4) After conventional capillary electrophoresis, the released tags are separated and detected as a fluorescence peak in an electropherogram, and results adjusted for surface area.

Immunohistochemistry (IHC) for peptide 3R in the skin samples can be performed, for example, on the Ventana Discovery XT system according to the manufacturer's instructions.

The quantification of the VeraTag assay reporter tags are performed by Monogram developed software, which recognizes fluorescent peaks from a capillary electrophoresis electropherogram. The concentration of the peptide 3R fluorescent tags is quantified by dividing by the sample area in mm² as determined above, and compared with the peptide 3R IHC staining intensity quantified by standard test categories.

Since the peptide 3R content can vary depending on age and UV exposure of the tissue, it is necessary to normalize the peptide 3R content of the slides to the tissue area (TA). Tissue area normalization can be verified by showing that the VeraTag peptide 3R signal increases linearly with sectional area on slides from the same sample. To evaluate the accuracy of the VeraTag peptide 3R assay, the peptide levels normalized to TA will be compared to IHC performed on other slides from the same sample. In some embodiments, the peptide 3R levels should show a continuous measurement over a dynamic range (>2 log₁₀ with HER2). Peptide 3R was represented with IHC histoscores from 0 to 3+ depending on the age and location of the samples, and the VeraTag assay could show a wide range as well.

Plotting the sun-protected areas together, and the sun-exposed areas together, could allow realization of the component of peptide 3R accumulation due to age alone, and that which is due to UV exposure.

In some embodiments, the assay is shown to provide a dynamic and continuous measurement of peptide 3R in skin. In some embodiments, the observed variability of the assay on replicate samples could be, for example, between about 10% to 20% over 10 assays by the same operator, or less than about 25%, 20%, 10%, 5%, or less in some cases.

Various other modifications, adaptations, and alternative designs are of course possible in light of the above teachings. Therefore, it should be understood at this time that within the scope of the appended claims the invention may be practiced otherwise than as specifically described herein. It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “taking a skin biopsy of a patient” includes “instructing the taking of a skin biopsy of a patient.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

What is claimed is:

1. A method of determining a patient's risk of developing a dermatologic condition, comprising the steps of:
receiving a skin biopsy sample of the patient, the skin biopsy sample comprising a skin marker, wherein the skin marker comprises peptide 3R;
providing a first binding partner conjugated to biotin, wherein the first binding partner is a first antibody;
providing a second binding partner conjugated to a fluorescent reporter tag, wherein the second binding partner is a second antibody;
exposing the first binding partner to the skin biopsy sample, such that the first binding partner binds to a first binding site of peptide 3R;
exposing the second binding partner to the skin biopsy sample, such that the second binding partner binds to a second binding site of peptide 3R;
exposing the skin biopsy sample to a molecular cleavage element such that the molecular cleavage element binds to the first binding partner, the molecular cleavage element comprising a photosynthesizer;
illuminating the tissue sample with light such that the molecular cleavage element causes the fluorescent reporter tag to dissociate from the second binding partner; and
quantitatively determining the amount of the fluorescent reporter tag released.

2. A method of determining the effect of therapy of a dermatological condition, comprising the steps of:
receiving a first skin biopsy sample of the patient, the first skin biopsy sample comprising a skin marker, wherein the skin marker comprises peptide 3R;
providing a first binding partner conjugated to biotin, wherein the first binding partner is a first antibody;
providing a second binding partner conjugated to a fluorescent reporter tag, wherein the second binding partner is a second antibody;
exposing the first binding partner to the first skin biopsy sample, such that the first binding partner binds to a first binding site of peptide 3R;
exposing the second binding partner to the first skin biopsy sample, such that the second binding partner binds to a second binding site of peptide 3R;
exposing the skin biopsy sample to a molecular cleavage element such that the molecular cleavage element binds to the first binding partner, the molecular cleavage element comprising a photosynthesizer;
illuminating the tissue sample with light such that the molecular cleavage element causes the fluorescent reporter tag to dissociate from the second binding partner; and
quantitatively determining the amount of the fluorescent reporter tag released to determine the amount of peptide 3R present in the first skin biopsy sample.

3. The method of claim 2, further comprising the steps of:
receiving a second skin biopsy sample of the patient, the second skin biopsy sample comprising a skin marker, wherein the skin marker comprises peptide 3R;
determining the amount of peptide 3R present in the second skin biopsy sample; and
comparing the amount of the peptide 3R present in the second skin biopsy sample with the amount of the peptide 3R present in the first skin biopsy sample,
wherein the second skin biopsy sample was taken from the patient at least about a month following the first skin biopsy sample.

4. A method, comprising:
performing a skin biopsy on a patient, wherein performing a skin biopsy comprises taking a skin biopsy sample comprising a skin marker, wherein the skin marker comprises peptide 3R;
ordering a diagnostic test on the skin biopsy sample, wherein the diagnostic test comprises the steps of:
receiving the skin biopsy sample of the patient;
providing a first binding partner conjugated to biotin, wherein the first binding partner is a first antibody;
providing a second binding partner conjugated to a fluorescent reporter tag, wherein the second binding partner is a second antibody;
exposing the first binding partner to the skin biopsy sample, such that the first binding partner binds to a first binding site of peptide 3R;
exposing the second binding partner to the skin biopsy sample, such that the second binding partner binds to a second binding site of peptide 3R;
exposing the skin biopsy sample to a molecular cleavage element such that the molecular cleavage element binds to the first binding partner, the molecular cleavage element comprising a photosynthesizer;
illuminating the tissue sample with light such that the molecular cleavage element causes the fluorescent reporter tag to dissociate from the second binding partner; and
quantitatively determining the amount of the fluorescent reporter tag released to determine the amount of peptide 3R present in the skin biopsy sample; and
analyzing the results of the amount of peptide 3R present in the skin biopsy sample.

5. The method of claim 4, further comprising the step of determining the risk of the patient developing a dermatologic condition.

6. The method of claim 5, wherein the dermatologic condition comprises a skin cancer.

7. The method of claim 6, wherein the skin cancer is a melanoma.

8. The method of claim 6, wherein the skin cancer is a basal cell carcinoma.

9. The method of claim 6, wherein the skin cancer is a squamous cell carcinoma.

10. The method of claim 4, further comprising the step of diagnosing a dermatologic condition.

11. The method of claim 10, wherein the dermatologic condition is selected from the group consisting of: melanoma, basal cell carcinoma, squamous cell carcinoma, actinic keratosis, xeroderma pigmentosum, albinism, xerosis, ichtyosis, keratoderma, dermatofibroma, dermatitis, acne, neurodermatitis, dermatitis herpetiformis, vitiligo, vasculitis, pemphigus, bullous pemphigoid, hyperkeratosis, eczema, psoriasis, rosacea, pityriasis rosea, warts, bacterial skin infection, viral skin infection, and a fungal skin infection.

12. The method of claim 4, further comprising the step of treating a dermatologic condition based upon the amount of peptide 3R present in the skin biopsy sample.

13. The method of claim 4, wherein the amount of peptide 3R present in the skin biopsy sample is a first amount of peptide 3R present in a first skin biopsy sample, and further comprising the step of comparing the first amount of peptide 3R with a second amount of peptide 3R present in a second skin biopsy sample of the patient.

14. The method of claim 13, wherein the second skin biopsy sample was taken at a time separated by at least about a week with respect to the first skin biopsy sample.

15. The method of claim 13, wherein the second skin biopsy sample was taken at a time separated by at least about a month with respect to the first skin biopsy sample.

16. The method of claim 13, further comprising the step of initiating therapy for a dermatologic condition after the comparing step.

17. The method of claim 13, further comprising the step of modifying therapy for a dermatologic condition after the comparing step.

18. The method of claim 13, further comprising the step of discontinuing therapy for a dermatologic condition after the comparing step.
